(19) United States
(12) Patent Application Publication
(10) Pub. No.: US 2023/0106497 A1
(43) Pub. Date: Apr. 6, 2023

(54) DRUG INFUSION DEVICES COMPATIBLE WITH DRUG FORMULATIONS

(71) Applicant: Medtronic, Inc., Minneapolis, MN (US)

(72) Inventors: Chandrasekhar R. Rajagopalan, Minneapolis, MN (US); Nicholas D. Stepka, Minneapolis, MN (US)

(21) Appl. No.: 17/799,541

(22) PCT Filed: Feb. 3, 2021
(86) PCT No.: PCT/US2021/016289

§ 371 Date: Aug. 12, 2022

Related U.S. Application Data
(60) Provisional application No. 62/979,100, filed on Feb. 20, 2020.

(51) Int. Cl. G16C 20/30; A61M 5/14; G01N 33/15

(52) U.S. Cl. CPC G16C 20/30; A61M 5/14; G01N 33/15; A61M 2207/00

(57) ABSTRACT

A method for manufacturing or selecting a medical device, such as a reservoir-based delivery device, for use in contact with a fluid composition, such as a drug formulation, includes considering one or more of: (i) the differential Hansen Solubility Parameter (HSP) value for and ingredient of the fluid composition and a component of a medical device; (ii) the concentration of the ingredient in the fluid composition relative to the maximum solubility of the ingredient in the fluid composition at the pH of the concentration; (iii) the potential for chemical reaction between the ingredient of the fluid composition and the component of the medical device; and (iv) baseline reactivity of the fluid composition with the component of the medical device.

### FIELD

The present technology is generally related to reservoir-based delivery devices, such as ambulatory or implantable infusion devices, and compatibility of such devices with drug formulations used with such devices.

### BACKGROUND

In many cases, existing injectable drug formulations configured for intravenous, intramuscular, or intrathecal delivery are adopted for delivery using reservoir-based delivery devices, such as ambulatory or implantable infusion devices. While such practices are generally safe for patients, the existing drug formulations may not be fully compatible with the delivery devices. For example, the drugs or other ingredients of the drug formulation may be taken up by one or more components of the delivery devices or may react with one or more components of the delivery devices.

Such interactions may reduce the useable lifespan of the delivery device. This may be particularly problematic with implantable reservoir-based delivery devices, which require an explant surgical procedure to replace the device.

### SUMMARY

The present disclosure describes methods for designing or selecting reservoir-based delivery devices or components having improved compatibility with drug formulations. The present disclosure also describes, as an example, a reservoir-based delivery device in which a component formed from silicone is replaced with a component formed from a fluoroelastomer.

The methods for manufacturing or selecting a medical device, such as a reservoir-based delivery device, for use in contact with a fluid composition, such as a drug formulation, may include considering one or more of: (i) the differential Hansen Solubility Parameter (HSP) value for and ingredient of the fluid composition and a component of a medical device; (ii) the concentration of the ingredient in the fluid composition relative to the maximum solubility of the ingredient in the fluid composition at the pH of the concentration; (iii) the potential for chemical reaction between the ingredient of the fluid composition and the component of the medical device; and (iv) baseline reactivity of the formulation medium with the component of the medical device.

In one aspect, the present disclosure describes a method for manufacturing or selecting a medical device, such as a reservoir-based delivery device, for use in contact with a fluid composition, such as a drug formulation. The method includes determining a differential Hansen Solubility Parameter (HSP) value for an ingredient of fluid composition and component of a medical device. If the differential HSP value is greater than 10, the medical device is manufactured or selected.

In another aspect, the present disclosure describes a method for manufacturing or selecting a medical device, such as a reservoir-based delivery device, for use in contact with a fluid composition, such as a drug formulation. The method includes (i) determining a differential Hansen Solubility Parameter (HSP) value for an ingredient of fluid composition and component of a medical device; and determining the maximum solubility of the ingredient at the pH of the fluid composition. If the differential HSP value is between 7 and 10 and if the concentration of the ingredient in the fluid composition is less than the maximum concentration for solubility at the pH of the composition, the medical device is manufactured or selected.

The concentration of the ingredient in the fluid composition relative to the maximum concentration for solubility at the pH of the composition may be proportional to the differential HSP value.

The concentration of the ingredient in the fluid composition may be less than 50% of the maximum concentration for solubility at the pH of the fluid composition.

In another aspect, the present disclosure describes a method for manufacturing or selecting a medical device, such as a reservoir-based delivery device, for use in contact with a fluid composition, such as a drug formulation. The method includes (i) determining a differential Hansen Solubility Parameter (HSP) value for an ingredient of fluid composition and component of a medical device; and determining the maximum solubility of the ingredient at the pH of the fluid composition. If the differential HSP value is below and if the concentration of the ingredient in the fluid composition is less than 50% of the maximum concentration for solubility at the pH of the fluid composition, the medical device is manufactured or selected.

The concentration of the ingredient in the fluid composition may be less than 25% of the maximum concentration for solubility at the pH of the fluid composition.

The details of one or more aspects of the disclosure are set forth in the accompanying drawings and the description below. Other features, objects, and advantages of the techniques described in this disclosure will be apparent from the description and drawings, and from the claims.

### BRIEF DESCRIPTION OF DRAWINGS

FIGS. 1-3 are flow diagrams that illustrate method for selecting a reservoir-based delivery device for use with a drug formulation.

### DETAILED DESCRIPTION

The present disclosure describes methods for designing or selecting reservoir-based delivery devices having improved compatibility with drug formulations. The present disclosure also describes, as an example, an implantable reservoir-based delivery device in which a component formed from silicone is replaced with a component formed from a fluoroelastomer. Such a change may result in improvements in compatibility of the delivery device and certain drug formulations, which may increase the useable lifespan of the infusion device.

For purposes of the present disclosure “reservoir-based delivery devices” are drug delivery devices that are designed to store a certain volume of a drug formulation in a reservoir and are configured to deliver the drug formulation over time to a target location of a patient. The reservoir-based delivery devices may be ambulatory or implantable. The reservoir-based delivery devices may be fixed-rate delivery devices or variable or programmable rate delivery devices. An example of a reservoir-based delivery device that is a variable or programmable rate delivery device is Medtronic, Inc.’s SynchroMed® II implantable infusion device.

For purposes of the present disclosure, reference to a compound will be understood to include reference to the compound, salts of the compound, solvates of the compound, and polymorphs of the compound. Examples of compounds that may be included in a drug formulation include the drug, preservatives, buffers, surfactants, and other excipients (e.g., ingredients) of the formulation.

Reservoir-based drug delivery devices may exhibit deviation from intended behavior as a result of interaction with drugs formulations. Interactions of the drug formulation with the device typically occur between an ingredient of the drug formulation and a polymeric component of the reservoir-based device that contacts the drug formulation. For example, components of the device comprising silicone elastomers may cause compatibility concerns with certain drug formulations.

The methods described herein may be used to design or select a reservoir-based drug delivery device having improved compatibility with a drug formulation. The methods may be employed with any suitable drug formulation comprising any suitable compound. Preferably the compound has a number-average molecular weight of less than 1000 Daltons.

The methods described herein provide guidance for selecting or designing a suitable reservoir-based delivery device for use with a drug formulation. The formulation may have a pH and concentration of ingredients. One or more of the following four factors may be considered to achieve compatibility between the reservoir-based delivery device and the drug formulation:
Differential Hansen Solubility Parameter (HSP) - based on HSP values of drug contacting materials of the reservoir-based delivery device and drug formulation ingredients
pH-solubility profile of ingredients of the drug formulation, such as the drug
Functional group - potential for chemical reaction between drug formulation ingredients and drug contacting materials of the reservoir-based delivery device
Baseline reactivity of formulation medium with drug contacting materials of the reservoir-based delivery device

These factors are discussed in additional detail below.

### Hsp

Without intending to be bound by theory, it is believed that HSP provides a thermodynamic indication of whether a compound will migrate into a component of the reservoir-based delivery device, such as a polymeric component of the reservoir-based delivery device. Substantial migration of the ingredient into a component of the reservoir-based delivery device may be considered an incompatibility of the drug formulation with the reservoir-based delivery device. Greater differences between HSPs of the compound and the component tend to result in lower amounts of migration.

For purposes of the present disclosure, differential HSP values of less than 7 are considered to be of high risk, HSP values between 7 and 10 are considered to be of medium risk, and HSP values of greater than 10 are considered to be of low risk for the drug to migrate into the component of the reservoir-based delivery device. Differential HSP values may be determined as described in Kitak, et al. (2015), “Determination of Solubility Parameters of Ibuprofen and Ibuprofen Lysinate,” Molecules, 20:21549-21568. One useful measurement of differential HSP values is described in Equation (4) of Kitak, et al. on page 21550. Other useful measurements include Equations (5), (6), and (7) of Kitak, et al. on pages 21550-21551.

If the HSP values present risks with one or more components of a particular reservoir-based delivery device, a reservoir-based drug delivery device having components formed from materials having HSP values with lower risk may be selected or designed.

### pH-Solubility Profile

Knowing the pH-solubility profile of an ingredient of a formulation in a solution; the pH of the drug formulation; and the concentration of the ingredient in the drug formulation will be helpful in designing or selecting a reservoir-based delivery device that is compatible with the drug formulation.

For example, if the concentration of the ingredient in the drug formulation is substantially below the maximum solubility of the ingredient at the pH of the drug formulation, the more likely that the drug formulation will be compatible with components of the reservoir-based delivery device. The further the ingredient concentration is below the maximum solubility concentration at the formulation pH, the more equilibrium will tend to drive the drug to remain in solution.

Accordingly, the HSP risk may be evaluated in the context of the concentration of the ingredient relative to the maximum solubility of the ingredient at the pH of the drug formulation.

### Functional Groups - Chemical Reaction

The presence of certain functional groups on ingredients of the formulation and certain functional groups on drug formulation contacting materials of the reservoir-based delivery devices may result in concern for chemical reaction between the formulation and the delivery device.

For example, silyl ether groups which may be present on silicone elastomer components of reservoir-based drug delivery device may react with amine groups, phosphate groups, or carboxylate groups of ingredients of the drug formulation.

The concern for potential chemical reaction may be evaluated in view of the HSP values and the concentration of the potentially problematic ingredients of the formulation relative to the maximum solubility of the potentially problematic ingredients at the pH of the formulation. For example, if the HSP risk is low and the concentration of the potentially problematic ingredients is low (relative to maximum solubility), the risk of incompatibility may be low despite potentially incompatible functional groups.

If the concern for chemical reaction is high, a reservoir-based drug delivery device having components formed from materials having less potentially incompatible functional groups may be selected or designed.

### Baseline Reactivity of Formulation Medium

In some cases, concerns of reactivity between the formulation without the drug and the formulation-contacting surfaces of the reservoir-based delivery device may exist. For example, if the formulation contains ingredients that may react with components of the reservoir-based delivery device at pH extremes, a reservoir-based delivery device having components that reduces such concerns may be selected or designed.

### Example of Substitution of Silicone Elastomer With Fluoroelastomer

With the above considerations in mind, an example in which one or more components of a reservoir-based delivery device formed from silicone elastomer is replaced with one or more components formed from a fluoroelastomer is described. Examples of components of a reservoir-based delivery device that may be formed from such elastomers include O-rings, tubing, and valves.

As discussed above, silicone elastomers comprise silyl ether groups which may react with amine groups, phosphate groups, or carboxylate groups of ingredients of the drug formulation. In contrast, fluoroelastomers comprise fluorocarbon groups, which may not be as reactive with amine groups, phosphate groups, or carboxylate groups. Accordingly, replacing a silicone elastomer with a fluoroelastomer may reduce potential chemical reactions with the delivery device component and an ingredient of the drug formulation.

In addition, silicone elastomers tend to have lower differential HSP values relative to ingredients of aqueous drug formulations typically used in reservoir-based delivery devices. In contrast, fluoroelastomers tend to have higher differential HSP values relative to ingredients of aqueous drug formulations.

Accordingly, and depending on the ingredients of the drug formulation, replacing a component formed from a silicone elastomer with a fluoroelastomer may provide benefits of reduced potential chemical reaction as well as reduced permeation and miscibility (higher differential HSP), which may result in increased useable life span of the reservoir-based delivery device.

A silicone elastomer may be replaced with any suitable fluoroelastomer. For example, the fluoroelastomer may be a fluorosilicone (partially or fully fluorinated) such as a liquid silicone rubber or a high consistency rubber fluorosilicone. One example of a fluorosilicone elastomer that may be used is MED50-5338 fluorosilicone liquid rubber available from NuSil Technology LLC (Carpinteria, CA USA). The fluoroelastomer may be a non-silicone fluoroelastomer such as those formed from FKM or FFKM monomers, either as a single or co-polymer. FKM is a family of fluoroelastomer materials defined by the ASTM International standard D1418-17 (Standard Practice for Rubber and Rubber Latices-Nomenclature). FKMs contain vinylidene fluoride as a monomer. Originally developed by DuPont (Viton®), FKMs are today also may be obtained from, for example, Daikin Chemical (Dai-El), 3M (Dyneon Fluoroelastomers), Solvay Specialty Polymers (Tecnoflon), and HaloPolymer (Elaftor). An example of a non-silicone FKM fluoroelastomers that may be used is DAI-EL T-530 available from Daikin America, Inc. (Orangeburg, NY USA). FFKMs (defined by ASTM 1418-17) containing a higher amount of fluorine than FKM fluoroelastomers.

Preferably, the component formed from a fluoroelastomer that replaces a component formed from a silicone elastomer has suitable mechanical properties to the silicone elastomers. Silicone elastomers may be crosslinked with permanent and temporary crosslinks, while untreated fluoroelastomers tend to not be cross-linked. The fluoroelastomers may be crosslinked by post-processing to have mechanical properties similar to silicone elastomers. For example, the fluoroelastomers may be crosslinked by chemical or electron beam crosslinking methods. Preferably, the fluoroelastomers comprise a combination of chemical and physical crosslinking as the silicone elastomers that they replace.

The fluoroelastomers may have any suitable hardness. For example, the fluoroelastomers may have a hardness in a range from 30 A durometer to 70 A durometer. The fluoroelastomers may be crosslinked to achieve a suitable hardness.

Using the principles described herein, one may also alter the formulation to be more compatible with the designed or selected reservoir-based delivery device. The teachings presented in U.S. Provisional Application No. 62/971,507, filed on Feb. 7, 2020, entitled DRUG FORMULATIONS FOR RESERVOIR-BASED DELIVERY, and having attorney docket no. A0003617US01, may be used to aid in preparing a suitable drug formulation. U.S. Provisional Application No. 62/971,507.

### Overviews of Selected Aspects

Overviews of selected aspects will be described with reference to the figures.

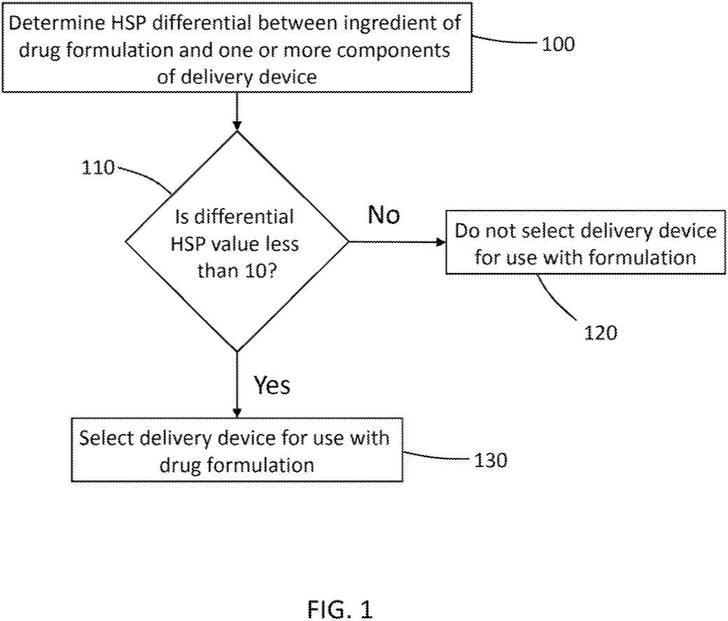

FIG. 1 illustrates a method for selecting a reservoir-based delivery device for use with a given drug formulation. First, the HSP differential between ingredients of the drug formulation and one or more components of the delivery device, such as drug formulation-contacting components of the delivery device, is determined (100). If the HSP value 10 or greater (110), the device is not selected for use with the formulation (120). If the HSP value is less than 10 (110), the device is selected for use with the formulation (130).

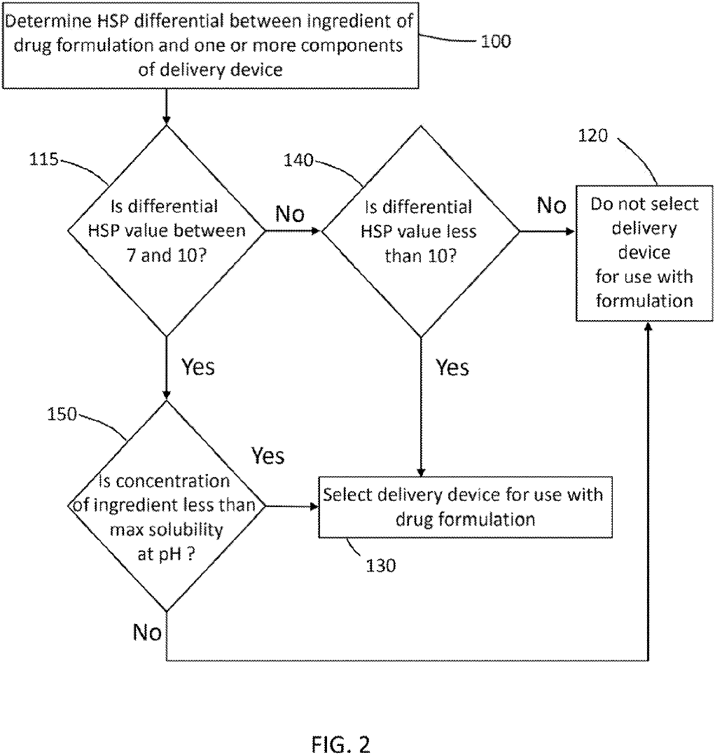

FIG. 2 illustrates an alternative method for selecting a reservoir-based delivery device for use with a given drug formulation. First, the HSP differential between ingredients of the drug formulation and one or more components of the delivery device, such as drug formulation-contacting components of the delivery device, is determined (100). If the HSP value is not between 7 and 10 (115) and the HSP is greater than 10 (140), the device is not selected for use with the formulation (120). If the HSP value is not between 7 and 10 (115) and the HSP is not greater than 10; i.e., is less than 7 (140), the device is selected for use with the formulation (130). If the HSP value is between 7 and 10 (115), a determination may be made as to whether the concentration of the ingredient in the formulation is less than the maximum solubility for the ingredient in the formulation at the formulation pH (150). If the concentration of the ingredient in the formulation is greater than the maximum solubility for the ingredient in the formulation at the formulation pH (150), the device is not selected for use with the formulation (120). If the concentration of the ingredient in the formulation is less than the maximum solubility for the ingredient in the formulation at the formulation pH (150), the device is selected for use with the formulation (130).

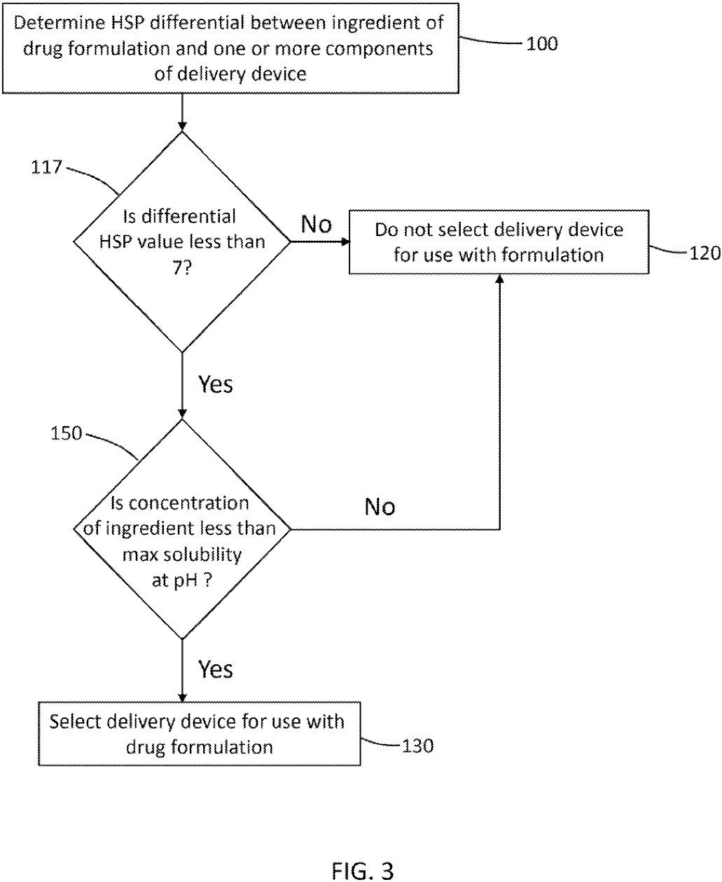

FIG. 3 illustrates an alternative method for selecting a reservoir-based delivery device for use with a given drug formulation. First, the HSP differential between ingredients of the drug formulation and one or more components of the delivery device, such as drug formulation-contacting components of the delivery device, is determined (100). If the HSP value is greater than 7 (117), the device is not selected for use with the formulation (120). If the HSP value is less than 7 (110) and the concentration of the ingredient in the formulation is greater than the maximum solubility for the ingredient in the formulation at the formulation pH (150), the device is not selected for use with the formulation (120). If the HSP value is less than 7 (110) and the concentration of the ingredient in the formulation is less than the maximum solubility for the ingredient in the formulation at the formulation pH (150), the device is selected for use with the formulation (130).

While the disclosure above relates mainly to compatibility of drug formulations with reservoir-based delivery devices, the concepts described herein may be used to determine compatibility of a drug formulation with a drug contacting material of any suitable medical device. Suitable medical devices include implantable infusion devices, ambulatory infusion devices, external infusion devices, catheters, syringes, and the like. Suitable infusion devices may comprise any suitable infusion mechanism, such as an osmotic pump, a peristaltic pump, a piston pump, propellant pump, and the like. Other suitable medical devices include those that may contact body fluid of a patient, such as leads, heart valves, blood pumps, implantable sensors, and the like.

The concepts described herein may also apply to contact of a medical device with any suitable fluid composition. Suitable fluid compositions include body fluid, such as blood, cerebral spinal fluid, interstitial fluid, urine, and the like.

It should be understood that various aspects disclosed herein may be combined in different combinations than the combinations specifically presented in the description and accompanying drawings. It should also be understood that, depending on the example, certain acts or events of any of the processes or methods described herein may be performed in a different sequence, may be added, merged, or left out altogether (e.g., all described acts or events may not be necessary to carry out the techniques). In addition, while certain aspects of this disclosure are described as being performed by a single module or unit for purposes of clarity, it should be understood that the techniques of this disclosure may be performed by a combination of units or modules associated with, for example, a medical device.

What is claimed is:

1. A method comprising:
determining a differential Hansen Solubility Parameter (HSP) value for an ingredient of fluid composition and component of a medical device;
manufacturing or selecting the medical device for use with the fluid composition if the differential HSP value is greater than 10.

2. A method comprising:
determining a differential Hansen Solubility Parameter (HSP) value for an ingredient of fluid composition and component of a medical device;
determining the maximum solubility of the ingredient at the pH of the fluid composition;
manufacturing or selecting the medical device for use with the fluid composition if the differential HSP value is between 7 and 10 and if the concentration of the ingredient in the fluid composition is less than the maximum concentration for solubility at the pH of the fluid composition.

3. The method of claim 2, wherein the concentration of the ingredient in the fluid composition relative to the maximum concentration for solubility at the pH of the fluid composition is proportional to the differential HSP value.

4. The method of claim 3, wherein the concentration of the ingredient in the fluid composition is less than 50% of the maximum concentration for solubility at the pH of the fluid composition.

5. The method of claim 2, wherein the concentration of the ingredient in the fluid composition is less than 50% of the maximum concentration for solubility at the pH of the composition.

6. The method of claim 1, wherein the medical device is a reservoir-based delivery device.

7. The method of claim 6, wherein the fluid composition is a drug formulation.

8. A method comprising:
determining a differential Hansen Solubility Parameter (HSP) value for an ingredient of fluid composition and component of a medical device;
determining the maximum solubility of the ingredient at the pH of the fluid composition;
manufacturing or selecting the medical device for use with the fluid composition if the differential HSP value is lower than 7 and if the concentration of the ingredient in the fluid composition is less than 50% of the maximum concentration for solubility at the pH of the fluid composition.

9. The method of claim 8, wherein the concentration of the ingredient in the fluid composition is less than 25% of the maximum concentration for solubility at the pH of the fluid composition.

10. The method of claim 8, further comprising determining whether the ingredient may have a potential for interaction with the component of the medical device.

11. The method of claim 10, wherein the medical device is manufactured or selected if it is determined that the potential for interaction is low.

12. The method of claim 11, wherein the medical device is manufactured or selected if it is determined that the potential for interaction is low when factoring in the relative concentration of the ingredient and the HSP value.

13. The method of claim 12, further comprising determining whether the ingredient may have a potential for interaction with the component of the medical device.

14. The method of claim 13, wherein the medical device is manufactured or selected if it is determined that the potential for interaction is low.

15. The method of claim 13, wherein the medical device is manufactured or selected if it is determined that the potential for interaction is low when factoring in the relative concentration of the ingredient and the HSP value.

16. The method of claim 8, further comprising determining whether the ingredient may have a potential for interaction with the component of the medical device.

17. The method of claim 16, wherein the medical device is manufactured or selected if it is determined that the potential for interaction is low.

18. The method of claim 16, wherein the medical device is manufactured or selected if it is determined that the potential for interaction is low when factoring in the relative concentration of the ingredient and the HSP value.

19. The method of claim 8, wherein the medical device is a reservoir-based delivery device.

20. The method of claim 19, wherein the fluid composition is a drug formulation.
